(19) United States
(12) Patent Application Publication
(10) Pub. No.: US 2024/0041980 A1
(43) Pub. Date: Feb. 8, 2024

(54) COMPOSITIONS AND METHODS FOR TREATING TYPE 2 DIABETES USING GRANULOCYTE-MACROPHAGE COLONY-STIMULATING FACTOR (GM-CSF)

(71) Applicant: THE REGENTS OF THE UNIVERSITY OF COLORADO, A BODY CORPORATE, Denver, CO (US)

(72) Inventors: Huntington POTTER, Denver, CO (US); Timothy BOYD, Johnstown, CO (US); Ching-Jung WANG, Aurora, CO (US)

(21) Appl. No.: 18/238,261

(22) Filed: Aug. 25, 2023

Related U.S. Application Data
(63) Continuation of application No. PCT/US2022/017845, filed on Feb. 25, 2022.
(60) Provisional application No. 63/159,380, filed on Mar. 10, 2021.
(60) Provisional application No. 63/154,546, filed on Feb. 26, 2021.

(51) Int. Cl. A61K 38/19; A61P 3/10; A61P 1/18

(52) U.S. Cl. CPC A61K 38/193; A61P 3/10; A61P 1/18

(57) ABSTRACT

Embodiments of the instant disclosure relate to novel methods for treatment of one or more pathologic entities of Type 2 diabetes. In certain embodiments, methods concern reducing the risk of developing, preventing and/or treating a pathologic entity of Type 2 diabetes or other condition having a pathologic entity component by administering to a subject in need thereof a granulocyte macrophage colony stimulating factor (GM-CSF) or recombinantly produced molecule or fragment thereof, or an analog thereof. In other embodiments, methods concern decreasing pancreatic islet amyloid deposition in a subject by administering to the subject, GM-CSF or recombinantly produced molecule or fragment thereof, or an analog thereof, wherein the subject has pancreatic islet amyloid deposition.

### PRIORITY

This U.S. Continuation Application claims priority to International Application PCT/US2022/017845, filed Feb. 25, 2022, which claims priority to U.S. Provisional Application No. 63/159,380 filed Mar. 10, 2021, and to U.S. Provisional Application No. 63/154,546 filed Feb. 26, 2021. These applications are incorporated herein by reference in their entireties for all purposes.

### FIELD

Embodiments of the instant disclosure relate to novel methods for treatment of one or more pathologic entities of Type 2 diabetes. In certain embodiments, methods concern preventing and/or treating a pathologic entity of Type 2 diabetes by administering to a subject in need thereof a granulocyte macrophage colony stimulating factor (GM-CSF) or an analog thereof or analogous agent thereof.

### BACKGROUND

Type 2 diabetes, also referred to as Type 2 diabetes mellitus (T2DM), is a metabolic disease with high blood glucose levels and glucose intolerance as typical clinical features of the disease due to defective insulin production/secretion by pancreatic beta cells ((β-cells) or insulin sensitivity. Metabolic syndrome encompasses several metabolically related symptoms; for example, obesity, hypertension, dyslipidemia, carbohydrate intolerance, and insulin resistance. When the body cannot produce enough insulin to override insulin resistance, blood sugar level increases, typically causing Type 2 diabetes in a subject having such symptoms. T2DM can also be characterized histopathologically by the presence and accumulation of fibrillary amyloid deposits in the pancreatic islets of Langerhans. Accumulation of pancreatic amyloid induces islet (β-cell apoptosis which leads to deficiency in beta-cell mass and overall islet dysfunction.

Islet dysfunction is an important pathophysiological mechanism of T2DM. The main manifestation of islet dysfunction is beta cells not being able to secrete enough insulin, which in turn leads to hyperglycemia, and typically T2DM. Islet amyloid formation reduces insulin-mediated glucose uptake and inhibits insulin secretion in beta cells. Therefore, increased pancreatic islet amyloid deposition plays an important role in the pathogenesis of T2DM. As such, development of therapies targeting pancreatic islet amyloid deposition are needed to protect beta-cell function in pancreatic islets and to attenuate the pathogenesis of T2DM.

There is no cure for T2DM currently. For patients with T2DM, the disease is usually managed with diet, exercise, and medications that are designed to help control blood sugar or that help their body use insulin more effectively. Accordingly, there is a significant population of T2DM patients having an unmet medical need for therapeutics that can not only treat current T2DM patients, but also inhibit key pathogenic processes in the pre-diabetic population to reduce onset or advancement of the disease.

### SUMMARY

Embodiments of the instant disclosure relate to novel compositions and methods for treating Type 2 diabetes. Other embodiments relate to novel compositions and methods for treating one or more pathologic entities of Type 2 diabetes in a subject in need thereof. In some embodiments, compositions and methods disclosed herein can reduce the onset of, prevent and/or treat Type 2 diabetes or a pathologic entity of Type 2 diabetes by administering to a subject in need thereof a composition including, but not limited to, granulocyte macrophage colony stimulating factor (GM-CSF), recombinantly produced molecule thereof, a mimetic or biologically active fragment thereof, or an analog thereof. In other embodiments, exogenous administration of a viral or plasmid vector or mRNA construct encoding GM-CSF or analog or mutant or mimetic thereof can be administered and expressed in the subject. In accordance with these embodiments, the subject can be diagnosed as having or suspected of developing Type 2 diabetes and/or having or suspected of developing a pathologic entity of Type 2 diabetes. In certain embodiments, the pathologic entity of Type 2 diabetes can be pancreatic islet amyloid deposition.

In certain embodiment, compositions disclosed herein can be used to treat or reduce pancreatic islet cell apoptosis. In some embodiments, a subject can be diagnosed as having or suspected of developing Type 2 diabetes and/or having or suspected of developing a pathologic entity of Type 2 diabetes. In certain embodiments, compositions disclosed herein containing GM-CSF, a recombinantly produced molecule thereof, a mimetic or biologically active fragment thereof, or an analog thereof, or exogenous administration of a viral or plasmid vector or mRNA construct encoding GM-CSF, an analog, a mutant, or biologically active fragment or mimetic thereof and subsequently expressed in the subject. In certain embodiments, compositions disclosed herein containing GM-CSF, a recombinantly produced molecule thereof, a mimetic or biologically active fragment thereof, or an analog thereof, or exogenous administration of a viral or plasmid vector or mRNA construct encoding GM-CSF, an analog, a mutant, or biologically active fragment or mimetic thereof and subsequently expressed in the subject can be administered to a subject to treat or reduce pancreatic islet cell apoptosis. In some embodiment, these compositions can reduce caspase 3 expression and/or activity in the pancreas of the subject.

In some embodiments, subjects contemplated herein can be a human (e.g. adult, adolescent, child, infant or fetus) or non-human animal such as a pet or livestock or other animal capable of developing Type 2 diabetes. In some embodiments, the subject is a horse, a dog, or a cat. In some embodiments, the subject has Type-2 diabetes or is suspected of being at risk of developing Type 2 diabetes. In certain embodiments, the subject has been diagnosed as having or is suspected of developing a pathologic entity as a result of having Type 2 diabetes. In some embodiments, compositions and methods disclosed herein can include administration of about 100 to about 400 μg/m²/day of GM-CSF, or about 150 to about 300 μg/m²/ day of GM-CSF, or about 250 μg/m²/day of GM-CSF, recombinantly produced molecule thereof or fragment or analog thereof in a single dose or multiple doses. In other embodiments, a recombinantly produced GM-CSF molecule, a mimetic or fragment thereof can be administered in a composition at a significantly lower concentration such as about 1.0 to about 200.0.μg/m²/day of GM-CSF or about 1.0 to about 150.0 μg/m²/day of GM-CSF or about 2.5 μg/m²/day to about 150 μg/m²/day of GM-CSF depending on potency and desired outcome, in a single, or multiple treatments in a day. In certain embodiments, recombinant GM-CSF can include, but is not limited to sargramostim, molgramostim, regramostim or other recombinant GM-CSF.

In other embodiments, exogenous administration of a viral or plasmid vector or mRNA construct encoding GM-CSF or GM-CSF fragment, or analog or mimetic within the body of the subject being treated can be administered for short term or prolonged treatment. In some embodiments, pancreatic islet amyloid depositions can be reduced in a subject receiving such a treatment compared to pancreatic islet amyloid depositions of the subject not receiving GM-CSF, a recombinantly produced molecule thereof or fragment or analog thereof In some embodiments, compositions and methods disclosed herein can stop the progression of or reduce the development of pancreatic islet amyloid deposition in the subject relative to a subject not receiving such a treatment. In other embodiments, compositions and methods disclosed herein for treating a Type 2 diabetes diagnosed subject with GM-CSF, a recombinantly produced molecule or fragment thereof, or an analog thereof can reduce or eliminate blood urea nitrogen (BUN) levels in the subject relative to a similar subject not receiving such a treatment. In some embodiments, compositions, and methods for administering GM-CSF, a recombinantly produced molecule or fragment thereof, or an analog thereof to a subject can increase serum amylase levels or concentrations in the subject relative to a subject not receiving such a composition.

In other embodiments, compositions and methods can include a composition including, but not limited to, GM-CSF, synthetic GM-CSF, recombinantly produced GM-CSF, naturally-occurring GM-CSF or a vector expressing GM-CSF, or fragment thereof, or an analog thereof formulated in a pharmaceutical composition, which can further include a pharmaceutically acceptable carrier or excipient. In certain embodiments, compositions and methods disclosed herein can include administration of a pharmaceutical composition including, but not limited to, GM-C SF, a recombinantly produced molecule or fragment thereof, or an analog thereof In accordance with these embodiments, the pharmaceutical composition can be administered to the subject by oral, intravenous, intranasal, subcutaneous, or other administration known in the art alone or in combination with other agents or for treating Type 2 diabetes in the subject.

In some embodiments, compositions and methods disclosed herein can be used to treat a subject diagnosed as having or suspected of developing pancreatic islet amyloid deposition that have at least one metabolic risk factor. In accordance with these embodiments, at least one metabolic risk factor can include, but is not limited to, abdominal obesity, hypertension, dyslipidemia, hyperinsulinemia, other metabolic factor or indicator: or a combination thereof In some embodiments, a subject contemplated herein can be pre-Type 2 diabetic.

In certain embodiments, kits of use for treating and/or preventing pancreatic islet amyloid deposition and/or treating Type 2 diabetes in a subject are contemplated. In some embodiments, kits disclosed herein can include a composition including but not limited to, GM-CSF, a recombinantly produced molecule or fragment thereof, or an analog thereof and at least one container.

### BRIEF DESCRIPTION OF THE DRAWINGS

FIGS. 1A-1D illustrate examples of quantification of thioflavin-S staining in a mouse pancreas as an indicator of pancreatic amyloid depositions (1A) illustrates an overview table and 1B-1D illustrate images depicting staining in accordance with certain embodiments of the instant disclosure.

FIGS. 2A-2C illustrate in 2A (treated) and 2B (control) representative stained images and 2C a bar graph plot of examples of pancreatic amyloid depositions of GM-CSF-treated mice compared to control untreated mice in accordance with certain embodiments of the present disclosure.

FIGS. 3A-3E represent bar graphs illustrating blood sample analyses for certain Type 2 diabetes-related biomarkers in GM-CSF-treated mice compared to negative control treated mice where 3A represents LDH levels; 3B represents ALT liver levels; 3C represents glucose levels; 3D represents BUN levels; and 3E represents amylase levels in accordance with certain embodiments of the present disclosure.

FIGS. 4A-4C represent fluorescent images analyzing the presence of caspase in pancreas samples with (4B) and without (4A) GM-CSF treatment and 4C represents caspase 3 levels illustrated by a bar graph of treated versus a control in accordance with certain embodiments of the present disclosure.

### DEFINITIONS

Terms, unless specifically defined herein, have meanings as commonly understood by a person of ordinary skill in the art relevant to certain embodiments disclosed herein or as applicable.

Unless otherwise indicated, all numbers expressing quantities of agents and/or compounds, properties such as molecular weights, reaction conditions, and as disclosed herein are contemplated as being modified in all instances by the term “about.” Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, rodents, sheep, goat, dog, cat, horse, cow, and the like. In some embodiments, the subject is a human such as an adult, adolescent, child, infant or fetus.

As used herein, “treatment,” “therapy,” “treatment regimen” and/or “therapy regimen” can refer to an intervention made in response to a condition, disease, disorder, or physiological condition manifested by a subject or to which a subject can be susceptible. The aim of treatment includes the alleviation or prevention of symptoms, slowing or stopping the progression or worsening of a condition, disease, disorder and/or the remission of the condition, disease, or disorder.

As used herein, “prevent,” “prevention,” “eliminate the risk” can refer to completely eliminating, or preventing, or delaying the onset of a particular disease condition, disorder or physiological condition related to the disease or condition, or to the reduction of the degree of severity of a condition related to a particular disease, disorder or physiological condition such as Type 2 diabetes, relative to the time and/or degree of onset or severity in the absence of intervention using compositions disclosed in certain embodiments disclosed herein.

The term “effective amount” or “therapeutically effective amount” can refer to an amount sufficient to effect beneficial or desirable biological and/or clinical results in a subject which can depend on age, severity of a condition, responsiveness to the agent and other factors.

“Development” or “progression” of a condition can mean initial manifestations and/or ensuing progression of the condition or worsening of the condition or side effects of the condition. Development of the condition or disease can be detectable and assessed using standard clinical techniques as well known in the art. However, development also refers to progression that can be undetectable. For purpose of this disclosure, development or progression refers to the biological course of the symptoms. “Development” includes, but is not limited to, occurrence, recurrence, and onset. As used herein “onset” or “occurrence” of a target disease or disorder includes initial onset and/or recurrence.

Unless otherwise defined, all technical terms used herein have the same meaning as commonly understood by one of ordinary skill in the art to which this disclosure belongs.

### DETAILED DESCRIPTION

In the following sections, certain exemplary compositions and methods are described to detail certain embodiments of the invention. It will be obvious to one skilled in the art that practicing the certain embodiments does not require the employment of all or even some of the specific details outlined herein, but rather that concentrations, times and other specific details can be modified through routine experimentation. In some cases, well known methods, or components have not been included in the description.

Embodiments of the instant disclosure relate to novel compositions and methods for treating Type 2 diabetes and compositions and methods for treating one or more pathologic entities of Type 2 diabetes. In some embodiments, compositions and methods disclosed herein can reduce the onset of, prevent and/or treat a pathologic entity of Type 2 diabetes by administering to a subject in need thereof a composition comprising granulocyte macrophage colony stimulating factor (GM-CSF), recombinantly produced molecule thereof or biologically active fragment thereof, or an analog thereof. In accordance with these embodiments, the subject can be diagnosed as having or suspected of developing a pathologic entity of Type 2 diabetes. In certain embodiments, the pathologic entity of Type 2 diabetes can include, but is not limited to, pancreatic islet amyloid deposition. In other embodiments, the pathological entity of a condition can include, but is not limited to, amyloid deposits related to a heart or kidney condition. In accordance with these embodiments, amyloid deposits of these conditions can include, but are not limited to, transthyretin amyloidosis and/or antibody deposits in any organ such as the heart, kidney, or other organ or body compartment. In certain embodiments, a condition can include, but is not limited to, systemic light chain amyloidosis, an overexpressed antibody light chain (LC) form fibril which deposit in organs and can cause failure of the organ. In some embodiments, compositions and methods disclosed herein can be used to treat, reduce onset of, or prevent Familial Amyloid Polyneuropathy (FAP), which includes amyloidosis diseases caused by, for example, pathogenic entities: transthyretin, apoprotein Al, and gelsolin. In some embodiments, compositions and methods disclosed herein can be used to treat, reduce onset of, or prevent Primary Systemic AL amyloidosis, which can be caused by pathogenic entities of amyloid fibrils made up of either heavy or light chain immunoglobulin fragments. In certain embodiments, compositions and methods disclosed herein can be used to treat or prevent AA amyloidosis, which can be caused by the pathogenic entity SSA. In some embodiments, compositions and methods disclosed herein can be used to treat or prevent β2M amyloidosis, which can be caused by a pathogenic entity of β2-microglobulin accumulation. In other embodiments, compositions and methods disclosed herein can be used to treat or prevent Senile systemic amyloidosis (SSA), which can be caused by pathogenic entities of wild-type, naturally-occurring or endogenous transthyretin protein. In some embodiments, compositions and methods disclosed herein can be used to treat or prevent organ-specific amyloidosis, for example, cutaneous amyloidosis. In accordance with these embodiments, compositions and methods disclosed herein can reduce the onset of, further progression of, prevent and/or treat a pathologic entity (e.g. amyloid deposits) by administering to a subject in need thereof a composition comprising GM-C SF, recombinantly produced molecule thereof or biologically active fragment thereof, or an analog thereof or construct or vector for expressing GM-CSF, recombinantly produced molecule thereof or biologically active fragment thereof to a subject.

In some embodiments, subjects can be a human or non-human animal capable of developing Type 2 diabetes or pathologic entity thereof or plaque build-up thereof In certain embodiments, subject can be a pet, horse or livestock. In some embodiments, the subject is a dog. In certain embodiments, the subject has been diagnosed as having or is suspected of developing a pathologic entity as a result of having Type 2 diabetes. In some embodiments, pancreatic islet amyloid depositions can be reduced in a subject receiving such a treatment compared to pancreatic islet amyloid depositions of the subject not receiving GM-CSF, a recombinantly produced molecule thereof or fragment or analog thereof. In some embodiments, compositions and methods herein can include administration of about 50 μg/m²/day to about 1000 μg/m²/day; about 100 μg/m²/day to about 750 μg/m²/day; about 150 μg/m²/day to about 600 μg/m²/day; about 200 μg/m²/day to about 500 μg/m²/day; about 200 μg/m²/day to about 400 μg/m²/day; or about 250 μg/m²/day of GM-CSF, recombinantly produced molecule thereof or fragment or analog thereof in a single treatment or multiple treatments per day. In other embodiments, a subject can be treated every other day, 2 times per week, once a week or other dosing regimen. In some embodiments, compositions and methods herein can include administration of about 250 μg/m²/day of GM-CSF, recombinantly produced molecule thereof or fragment or analog thereof in a single dose or multiple doses. In some embodiments, two doses can be provided to the subject where the total concentration of GM-CSF or analog thereof is about 50 μg/m²/day to about 1000 μg/m²/day or about 100 μg/m²/day to about 500 μg/m²/day or about 200 μg/m²/day to about 300 μg/m²/day or about 250 μg/m²/day. In certain embodiments, recombinant GM-CSF can include, but is not limited, sargramostim, molgramostim, regramostim or other recombinant GM-CSF. In other embodiments, exogenous administration of a viral or plasmid vector or mRNA construct designed to encode GM-C SF within the body of the subject being treated can be administered for short term or prolonged treatment. In some embodiments, a recombinantly produced molecule or fragment thereof can be administered in a composition at a significantly lower concentration such as about 2.5 ug/m²/day to about 500 ug/m²/day of GM-CSF.

In some embodiments, compositions, and methods for treating pancreatic islet amyloid depositions can be used to reduce pancreatic islet amyloid deposits in a subject receiving such a treatment compared to pancreatic islet amyloid depositions of the subject not receiving GM-CSF, a recombinantly produced molecule thereof or fragment or analog thereof In some embodiments, compositions and methods disclosed herein can stop the progression of or reduce the development of pancreatic islet amyloid deposition in the subject relative to a subject not receiving such a treatment. In other embodiments, compositions and methods disclosed herein for treating a Type 2 diabetes diagnosed subject with GM-CSF, a recombinantly produced molecule or fragment thereof, or an analog thereof can reduce or eliminate blood urea nitrogen (BUN) levels in the subject relative a similar subject not receiving such a treatment.

In some embodiments, compositions, and methods for administering GM-CSF, a recombinantly produced molecule or fragment thereof, or an analog thereof to a subject can increase serum amylase levels or concentrations in the subject relative to a subject not receiving such a composition. In certain embodiments, the GM-CSF is recombinantly produced and in certain embodiments, the recombinantly produced GM-CSF can include, but is not limited to, sargramostim, molgramostim, regramostim or other recombinant GM-CSF.

In other embodiments, compositions and methods disclosed herein can include a composition including, but not limited to, GM-CSF, a recombinantly produced molecule or fragment thereof, or an analog thereof formulated in a pharmaceutical composition, which can further include a pharmaceutically acceptable carrier or excipient. In certain embodiments, compositions and methods disclosed herein can include administration of a pharmaceutical composition including, but not limited to, GM-CSF, a recombinantly produced molecule or fragment thereof, mimetic known in the art thereof, or an analog thereof or a vector encoding GM- CSF, a recombinantly produced molecule or fragment thereof, a mimetic thereof or analog thereof. In certain embodiments, the GM-CSF is recombinantly produced and in other embodiments, the recombinantly produced GM-CSF can include, but is not limited to, sargramostim, molgramostim, regramostim or other recombinant GM-CSF. In other embodiments, administration of a viral, mammalian, plasmid or other vector or mRNA construct encoding GM-C SF, a recombinantly produced molecule or fragment thereof, a mimetic thereof, or an analog thereof within the body of the subj ect being treated can be administered for short term, predetermined or prolonged treatment. In accordance with these embodiments, the pharmaceutical composition can be administered to the subject by any method known in the art. In other embodiments, the pharmaceutical composition can be administered to the subj ect by intravenous, intranasal, intra-arterially, orally, subcutaneously, by slow-release microparticles or timed-released formulation, by targeted deposit directly to the pancreas or other organ or tissue harboring deposits or by subcutaneous administration. In yet other embodiments, the pharmaceutical composition can be administered to the subj ect by intravenous and/or subcutaneous administration. In some embodiments, the subject can be treated one time, two times, or three times daily for a predetermined time to reduce and or prevent progression of, pancreatic islet amyloid deposition or other deposition in the subject contemplated herein. In yet other embodiments, the pharmaceutical composition can be administered to the subject alone, in combined treatment regimens or in combination with other agents or treatments for ameliorating the symptoms of, side effects of, or the condition of Type 2 diabetes in the subject. In some embodiments, compositions and methods disclosed herein can be used in combination treatments to reduce onset, prevent, reduce progression of and/or treating a pathological entity of Type 2 diabetes where other treatments can include any standard treatment for the condition. For example, it is contemplated to be used in combination with dietary constraints, dialysis or other known treatment of the condition (e.g., Type 2 diabetes) which will be beneficial or potentially synergistic to treat the condition. In certain embodiments, standard Type 2 diabetes treatment regimens can be combined with administering a composition including, but not limited to, GM-CSF a recombinantly produced molecule or fragment thereof, a mimetic thereof, or an analog thereof disclosed herein as differing approaches to treat the same condition for improved outcomes. In certain embodiments, treatment regimens can be alternated with standard treatments. In other embodiment, diet, regular exercise and other Type 2 diabetes treatments can be used at the same time as administering a composition disclosed herein to a sub j ect.

In some embodiments, compositions and methods disclosed herein can be used to treat a subject diagnosed as having or suspected of developing pancreatic islet amyloid deposition that further have at least one metabolic risk factor indicator. In accordance with these embodiments, at least one metabolic risk factor can include, but is not limited to, abdominal obesity, hypertension, dyslipidemia, hyperinsulinemia, other metabolic factor or indicator, or a combination thereof. In some embodiments, a subject contemplated herein can be pre-Type 2 diabetic. In yet other embodiments, a subject can have Type 2 diabetes but be pre-pancreatic islet amyloid deposition. In other embodiments, a subject can have early stages of Type 2 diabetes, middle stages of Type 2 diabetes or advanced stage Type 2 diabetes having mild or severe conditions of, or suspected of developing, pancreatic islet amyloid depositions.

Type 2 diabetes can be characterized by fibrillary amyloid deposits in the pancreatic islets of Langerhans, and which the pancreatic amyloid induces islet (3-cell apoptosis that can lead to deficient (3-cell mass. It has been found that increased islet amyloid severity is negatively correlated with insulin-positive area per islet. The pancreatic amyloid deposits in Type 2 diabetes are principally made up of human islet amyloid polypeptide (hIAPP), also known as human amylin. IAPP/amylin is a 37-residue polypeptide hormone that is expressed almost exclusively in pancreatic islet (3-cells, and which is co-produced and co-secreted with insulin in a molar ratio of about 1:100 in healthy subjects, but which occurs in a ratio of about 1:20 in Type 2 diabetic subjects. This decrease in circulating amylin in Type 2 diabetes occurs concurrently with increasing pancreatic amyloid accumulation.

In certain embodiments, the present disclosure can include kits of use for treating and/or preventing pancreatic islet amyloid deposition in a subject. In some embodiments, kits disclosed herein can include a composition including but not limited to, GM-CSF, a recombinantly produced molecule or fragment thereof, or an analog thereof and at least one container. In some embodiment, a kit of use to treat diabetes or other adverse deposit disclosed herein can include sargramostim, molgramostim, regramostim or other recombinant GM-C SF or a viral or plasmid vector or mRNA construct designed to encode GM-CSF within the body of the subject being treated can be included. In other embodiments, kits disclosed herein can further include at least one device for use in collecting a baseline blood sample or other body fluid or test sample from a subject, analyzing a baseline blood sample or other body fluid or test sample from a subject, collecting a follow-up blood sample or other body fluid or test sample from a subject, analyzing a follow-up blood sample or other body fluid or test sample from a subject, or a combination thereof. In certain embodiments, blood samples or other body fluid or test sample collected from a subject can be used to assess levels of the treating agent (e.g., GM-CSF) in the subject to assess whether to increase/decrease the frequency or dose of the treating agent or change the dosing regimen to better suit the subject being treated. In other embodiments, blood samples or other body fluid or test sample collected from a subject can be used to assess levels of pancreatic islet amyloid deposition in a subject before, during and/or after treatment. In yet other embodiments, blood samples or other body fluid or test sample collected from a subject can be used to assess treatment regimen in relation to the level of GM-CSF, a recombinantly produced molecule or fragment thereof, a mimetic thereof, or an analog thereof and assess levels of pancreatic islet amyloid deposition in a subject before, during and/or after treatment. In some embodiments, treatments can be increased in dosing regimen, frequency and/or concentration based on analysis of one or more sample from a subject.

Embodiments of the instant disclosure relate to novel compositions and methods for treatment of pancreatic islet amyloid deposition. In accordance with these embodiments, pancreatic islet amyloid deposition can occur or be suspected of developing in a subject having a metabolic syndrome. As used herein the term “metabolic syndrome” can refer to a group of risk factors and refer to a subject's risk for coronary artery disease, heart failure (HF) (also referred to herein as congestive heart failure (CHF)), stroke, Type 2 diabetes, or a combination thereof. In some embodiments, a subject having a metabolic syndrome can have one or more metabolic risk factors. In some embodiments, a subject suited for methods herein can have one or more metabolic risk factors including, but not limited to, abdominal obesity, hypertension, dyslipidemia, hyperinsulinemia, or any combination thereof. Abdominal obesity can be a metabolic risk factor for a subject herein who has waist to hip ratio greater than about 0.90. The term “hypertension” as used herein can refer to a blood pressure higher than a normal blood pressure for a subject. In some embodiments, normal blood pressure for a human subject herein can be less than about 120 mm Hg for systolic and/or less than about 80 mm Hg for diastolic pressure. In some embodiments, a human subject having a blood pressure greater than about 130 mm Hg for systolic and/or greater than about 85 mm Hg for diastolic pressure can be diagnosed as having hypertension.

In certain embodiments, the term “dyslipidemia” as used herein can refer to a subject having high levels of plasma cholesterol, high levels of plasma triglycerides, low levels of plasma a high-density lipoprotein (HDL) cholesterol level, or any combination thereof In some embodiments, a human subject disclosed herein having a triglyceride level of about 150 mg/dL or above or about 150 to about 199 or about 200 or above can be consider as having high or very high levels of triglycerides. In some embodiments, 150 mg/dL triglycerides and below can be considered as normal levels. In certain embodiments, a HDL cholesterol level below about mg/dL or below about 50 mg/dL or below about 40 mg/dL can be considered at risk for having or developing dyslipidemia. In yet other embodiments, a human subject having both a triglyceride level above about 150 mg/dL or more and an HDL cholesterol level below about 40 to about 60 mg/dL can be diagnosed as having dyslipidemia. These threshold levels can vary depending on the subject and specifics regarding the subject such as sex, age, weight, and other relevant parameters of the subject.

As used herein, the term “hyperinsulinemia” can refer to a subject having excess levels of insulin circulating in the blood relative to the level of glucose circulating in the blood. In some embodiments, a human subject having a fasting blood sugar level above about 99 mg/dL or about 100 mg/dL or up to about 125 mg/dL or above can be diagnosed as having hyperinsulinemia. In some embodiments, a human subject having a blood glucose level higher than about 30 mg/dL after 1 mg of glucagon is administered intravenously can be diagnosed as having hyperinsulinemia. In some embodiments, evaluation in a subject of glycemic response to injection of glucagon at the end of the fast or during a spontaneous hypoglycemic event can provide a rapid, reliable measure of increased insulin action and can be helpful in establishing the diagnosis of hyperinsulinemic hypoglycemia. An increase in plasma glucose concentration of greater than 30 mg/dL (1.7 mmol/L) in response to administration of glucagon (e.g., 1 mg intravenously or intramuscularly) indicates inappropriate conservation of liver glycogen during hypoglycemia and indicates suppression of liver glycogenolysis by excessive insulin action. It is contemplated herein that a nonhuman subject, depending on the subject, can be treated with similar regimens as a human with considerations to the specifics of the subject such as age, weight, size and condition to be treated such as early signs of the condition to advanced stages of the condition (e.g., Type 2 diabetes).

In some embodiments, pancreatic islet amyloid deposition can occur or be suspected of developing in a subject having at least one metabolic disorder. The term “metabolic disorder,” as used herein can refer to disorders, conditions, or diseases that result in perturbation of the normal physiological state of homeostasis due to an alteration in metabolism (anabolism and/or catabolism). An alteration in metabolism can result from an inability to break down (catabolize) a substance that should be broken down (e.g., phenylalanine) and as a result the substance and/or an intermediate substance builds up to toxic levels, or from an inability to produce (anabolize) some essential substance (e.g., insulin). In accordance with these embodiments, a subject having at least one metabolic disorder can have diabetes (also referred to as diabetes mellitus or “DM”). Diabetes mellitus (DM) is a chronic metabolic disorder characterized by high blood sugar (glucose) levels which result from defects in insulin secretion or action, or both. DM can be broadly classified into three types by etiology and clinical presentation, including type 1 diabetes, Type 2 diabetes, and gestational diabetes (GDM). Less common types of diabetes can include monogenic diabetes and secondary diabetes.

In some embodiments, pancreatic islet amyloid deposition can occur or develop in a subject having Type 2 diabetes. Type 2 diabetes is also known as insulin-resistant diabetes, non-insulin dependent diabetes, adult-onset diabetes, or Type 2 diabetes mellitus (T2DM). In T2DM, beta cells of the pancreas can produce insulin, at least in the early stages of the disease, but the body is unable to use it effectively because cells become resistant to the action of insulin. In later stages of the condition, beta cells can stop producing insulin which leads to multiple adverse conditions and often death of the subject.

In some embodiments, pancreatic islet amyloid deposition can occur or develop in a subject having pre-Type 2 diabetes. As used herein, the term “pre-Type 2 diabetic,” can refer to one or more early diabetic conditions. Examples of pre-Type 2 diabetic conditions can include, but are not limited to, impaired glucose utilization, abnormal or impaired fasting glucose levels, impaired glucose tolerance, impaired insulin sensitivity and insulin resistance. Pre-Type 2 diabetes can also be characterized by higher-than-normal hemoglobin Al c level (e.g., between and 6.4% in hemoglobin Al c test). Pre-Type 2 diabetes can be diagnosed by various blood tests, such as hemoglobin Al c test, fasting plasma glucose (FPG) test and oral glucose tolerance test (OGTT).

In some embodiments, pancreatic islet amyloid deposition can occur or develop in a subject having or developing a metabolic disorder and further include at least one metabolic risk factor. In some embodiments, pancreatic islet amyloid deposition can occur develop in a subject having or suspected of developing pre-Type 2 diabetes and further include at least one metabolic risk factor. In some embodiments, pancreatic islet amyloid deposition can occur or develop in a subject having or suspected of having T2DM and further include at least one metabolic risk factor. In accordance with these embodiments, metabolic risk factors include any metabolic risk factor associated with these conditions known in the art.

Embodiments disclosed herein concern treatment of one or more pathologic entities of Type 2 diabetes for improved outcome and reduced progression of the disease or condition. In some embodiments, methods of treating pathologic entity of Type 2 diabetes for improved outcome can include, but is not limited to, treating or reducing the presence or accumulation of pancreatic islet amyloid deposits. In certain embodiments, a subject having or suspected of developing a pancreatic islet amyloid deposition can be treated by compositions disclosed herein.

Embodiments disclosed herein concern prevention of forming one or more pathologic entities of Type 2 diabetes for improved outcome. In some embodiments, methods of preventing a pathologic entity of Type 2 diabetes for improved outcome can be reducing the risk of, onset of, progression of, or preventing pancreatic islet amyloid deposition or other amyloid deposition. In certain embodiments, a subject suspected of developing pancreatic islet amyloid deposition can be treated by compositions disclosed herein to reduce the onset of, risk of, or prevent pancreatic islet amyloid deposition. In accordance with these embodiments, a subject suspected of developing a pancreatic islet amyloid deposition can include a subject having a greater number of metabolic risk factors associated with the subject's condition. In accordance with these embodiments, a subject suspected of developing a pancreatic islet amyloid deposition can have or be suspected of developing a metabolic syndrome. In accordance with these embodiments, a subject suspected of developing pancreatic islet amyloid deposition can have or be suspected of developing insulin resistance. In other embodiments, a subject suspected of developing a pancreatic islet amyloid deposition can have or be suspected of developing pre-Type 2 diabetes. In some embodiments, a subject suspected of developing a pancreatic islet amyloid deposition can have or be suspected of developing Type 2 diabetes with or without vascular complications. Vascular complications can include, but are not limited to, retinopathy, nephropathy, neuropathy, peripheral vascular disease, ischemic heart disease, atherosclerosis, myocardial infarction, stroke, related microvascular or macrovascular events, or any combination thereof In certain embodiments, a male subject suspected of developing a pancreatic islet amyloid deposition can have or be suspected of developing erectile dysfunction. In other embodiments, a subject suspected of developing a pancreatic islet amyloid deposition can have or be suspected of developing Type 2 diabetes with venous insufficiency or venous disease. Venous insufficiency or disease complications can include, but is not limited to, diabetic neuropathic foot, venous ulcer disease, venous thromboembolism, varicose veins, related venous events, or any combination thereof. Venous insufficiency can lead to devastating consequences in a subject experiencing these conditions such as amputation of feet and limbs if the condition is severe.

In certain embodiments, methods for treating and/or preventing a pathologic entity of Type 2 diabetes (e.g., pancreatic islet amyloid deposition) can include administering GM-CSF, recombinant, mimetic or an analog thereof In some embodiments, islet amyloid deposition can be a pathogenic feature of Type 2 diabetes, and these deposits can contain amyloidogenic peptide islet amyloid polypeptide (LAPP), also referred to as amylin.

In some embodiments, a GM-CSF suitable for use disclosed herein can be a native GM-CSF, a recombinant GM-CSF, a mimetic, an analog or a recombinant variant of the GM- CSF. In accordance with these embodiments, a native GM-CSF can be a human GM-CSF purified native molecule according to methods known in the art. In some embodiments, a GM- CSF mimetic is a molecule that retains GM-CSF activities such as a GM-CSF fragment construct or similar. In accordance with these embodiments, a recombinant GM-CSF and/or a recombinantly produced variant of the GM-CSF can be obtained using polynucleotides encoding GM-CSF and/or GM-CSF variants for example, using cell-free expression systems such as reticulocyte lysate based expression systems, or by standard recombinant expression systems. In some embodiments, polynucleotides encoding GM-CSF and/or GM-CSF variants or analogs or mimetics thereof can be cloned into expression vectors and expressed using standard procedures. In some other embodiments, recombinant GM-CSF and/or GM-CSF variants or analogs or mimetics thereof can be purified using known methods in the art, including but not limited to chromatography. In some embodiments, a recombinant human GM- CSF and/or a recombinant variant of the human GM-CSF or analogs or mimetics thereof can be obtained from polynucleotides encoding human GM-CSF and/or human GM-CSF variants. In some embodiments, a GM-CSF suitable for use herein can be a native human GM-CSF, a recombinant human GM-CSF, or a recombinant variant of human GM-CSF or a mimetic thereof or a recombinantly produced mimetic thereof In certain embodiments, recombinant GM-CSF can include sargramostim, molgramostim, regramostim or other recombinant GM-CSF. In other embodiments, exogenous administration of a viral or plasmid vector or mRNA construct designed to encode GM-C SF within the body of the subject being treated can be administered for short term, timed or prolonged treatment using genetically engineered constructs.

In certain embodiments, GM-C SF suitable for use herein can be formulated to form a pharmaceutical composition, which can further include a pharmaceutically acceptable carrier, diluent or excipient. In some embodiments, any of the pharmaceutical compositions to be used in the present methods can include pharmaceutically acceptable carriers, excipients, or stabilizers in the form of lyophilized formations or aqueous solutions. In some embodiments, known agents to treat Type 2 diabetes can be combined or mixed with a composition containing GM-C SF, a recombinant molecule thereof, an analog thereof or mimetic thereof

In some embodiments, pharmaceutical compositions or formulations herein can be for parenteral administration. In accordance with these embodiments, pharmaceutical compositions or formulations herein can be for intramuscular (IM) administration, subcutaneous (SC) administration, intravenous (IV) administration, or any combination thereof. In other embodiments, a time-release composition can be used to treat the subject over a predetermined period of time with reduced subject interfacing and time-released agent for a predictable treatment regimen of the subject.

In certain embodiments, formulations herein suitable for parenteral administration can include aqueous and non-aqueous sterile injection solutions which can contain anti- oxidants, buffers, bacteriostats and solutes which render the formulation isotonic with the blood of the intended recipient; and aqueous and non-aqueous sterile suspensions which can include suspending agents and thickening agents. Aqueous solutions can be suitably buffered (preferably to a pH of from about 3 to about 9). The preparation of suitable parenteral formulations under sterile conditions is readily accomplished by standard pharmaceutical techniques well known to those skilled in the art.

In certain embodiments, pharmaceutical compositions herein to be used for in vivo administration should be sterile. This can be readily accomplished by, for example, filtration through sterile filtration membranes. Sterile injectable solutions are generally prepared by incorporating hydrogels in the required amount in the appropriate solvent with various other ingredients enumerated above, as required, followed by filter sterilization. Generally, dispersions can be prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

In certain embodiments, pharmaceutical compositions disclosed herein can also include other ingredients or agents such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins, chelating agents such as EDTA; sugar alcohols such as mannitol or sorbitol; salt-forming counterions such as sodium; and/or nonionic surfactants such as Tween, pluronics or polyethylene glycols.

In certain embodiments, any GM-CSF, for example, native GM-CSF, recombinant GM-CSF, mimetic thereof, analog thereof or a recombinant variant of GM-CSF can be used for treating and/or preventing a pathologic entity of Type 2 diabetes (e.g., pancreatic islet amyloid deposition). In some embodiments, methods herein can be used for treating and/or preventing a pathologic entity of Type 2 diabetes (e.g., pancreatic islet amyloid deposition) in a subject in thereof of by administering a GM-CSF described herein, as well as a pharmaceutical composition having such. In some embodiments, methods disclosed herein can include administration of an effective concentration of GM-CSF or a pharmaceutical composition to a subject who needs treatment via a suitable route (e.g., intravenous infusion, subcutaneous injection, oral administration) at a suitable concentration as disclosed herein. In some examples, GM-CSF or a pharmaceutical composition herein can be administered to a subject by injection through a syringe, a catheter, a trocar, a cannula, a microneedle, a continuous portable infusion method and the like. In some embodiments, methods of administering GM-CSF or a pharmaceutical composition herein can include application of a dissolvable GM-CSF-incorporated microneedle patch, intradermal patch and/or array onto the skin. In some embodiments, GM-CSF or a pharmaceutical composition disclosed herein can be administered by continuous infusion by a portable infusion pump.

In certain embodiments, effective concentrations of GM-CSF or a pharmaceutical composition disclosed herein can vary, as recognized by those skilled in the art, depending on route of administration, excipient usage, and co-usage with other active agents. For example, an “effective amount” of GM-CSF, such as sargramostim, can be the amount that alone, or together with further doses, produces the desired response, e.g., alleviating one or more symptoms associated with a pathologic entity of T2DM, stabilizing (e.g. not worsening) the state of T2DM progression, delay or slowing of T2DM progression, or any combination hereof. In accordance with these embodiments, an “effective amount” of GM-C SF (e.g., sargramostim) or similar can be an amount that can alleviate one or more symptoms associated with a pathologic entity of Type 2 diabetes including beta cell dysfunction, insulin resistance, hyperglycemia, elevated blood urea nitrogen levels (BUN), lowered serum amylase levels, or any combination thereof. In some embodiments, such responses to an effective amount of GM-CSF (e.g., sargramostim) can be monitored by routine methods or can be monitored according to the methods disclosure herein.

In some embodiments, the desired responses to treatment of a target pathologic entity of Type 2 diabetes (e.g., pancreatic islet amyloid deposition) can also include delaying the onset or progress of one or more conditions associated with advanced T2DM. Examples of conditions associated with advanced T2DM can include, but are not limited to, macrovascular disease (e.g. myocardial infarction, stroke), microvascular disease (e.g. retinopathy, nephropathy), neuropathy, and the like.

In some embodiments, compositions and methods disclosed herein can include administration of an effective concentration of GM-CSF or a pharmaceutical composition thereof to a subject in need thereof resulting in a therapeutically or prophylactically significant change in one or more clinically detectable markers of T2DM by at least about 1% to about 100% (e.g., about 1%, about 5%, about 10%, about 20%, about 30%, about 40%, about 50%, about 60%, about 70%, about 80%, about 90%, up to 100%) as compared to a control or non- treated subject or the state of the subject prior to administering the compositions described herein. Clinically detectable markers of T2DM can be measured by a glycated hemoglobin (AIC) test, a random blood sugar test, a fasting blood sugar test, an oral glucose tolerance test (OGTT), or the like. It is understood, however, that the total daily usage of the compositions and formulations as disclosed herein can be decided by the attending physician or other health professional within the scope of sound medical judgment. The exact amount required will vary depending on factors such as the type of disease being treated, gender, age, and weight of the subject.

In some embodiments, methods disclosed herein can include administration of an effective concentration of GM-C SF or a pharmaceutical composition to a subject in need thereof resulting in a therapeutically or prophylactically significant reduction in one or more symptoms associated with a pathologic entity of Type 2 diabetes (e.g., beta cell dysfunction, insulin resistance, hyperglycemia, elevated blood urea nitrogen levels (BUN), lowered serum amylase levels) by at least about 1% to about 100% (e.g., about 1%, about 5%, about 10%, about 20%, about 30%, about 40%, about 50%, about 60%, about 70%, about 80%, about 90%, up to 100%) compared to a control or un-treated subject or the state of the subject prior to administering the compositions described herein.

In some embodiments, methods disclosed herein can include administration of an effective concentration of GM-C SF or a pharmaceutical composition to a subject in need thereof resulting in a therapeutically or prophylactically significant delay of the onset or progress of one or more conditions associated with advanced Type 2 diabetes (e.g., myocardial infarction, stroke, retinopathy, nephropathy, neuropathy) or other condition contemplated herein having an adverse deposit disclosed herein, by at least about 1% to about 100% (e.g., about 1%, about 5%, about 10%, about 20%, about 30%, about 40%, about 50%, about 60%, about 70%, about 80%, about 90%, up to 100%) compared to a control or non-treated subject or the state of the subject prior to administering the compositions described herein.

In certain embodiments, methods of administering GM-CSF or a pharmaceutical composition to a subject in need thereof can reduce or prevent beta-cell injury in the pancreas, prevent beta-cell death (i.e., beta-cell apoptosis) in the pancreas, or a combination thereof In some embodiments, methods disclosed herein can include administration of an effective concentration of GM-C SF or a pharmaceutical composition to a subject in need thereof resulting in about 1% up to 100% (e.g., about 1%, about 5%, about 10%, about 20%, about 30%, about 40%, about 50%, about 60%, about 70%, about 80%, about 90%, up to 100%, all percentages in between are included) increase or preservation of viable beta-cells compared to a control or un- treated subject or the state of the subject prior to administering the compositions described herein. In some embodiments, methods disclosed herein can include administration of an effective concentration of GM-CSF or analog, mimetic or biologically active fragment or a pharmaceutical composition thereof to a subject in need thereof resulting in about 1% up to 100% (e.g., about 1%, about 5%, about 10%, about 20%, about 30%, about 40%, about 50%, about 60%, about 70%, about 80%, about 90%, up to 100%, all percentages in between are included) increase or preservation of viable beta-cells compared to a control or un-treated subject or the state of the subject prior to administering the compositions disclosed herein.

In some embodiments, methods disclosed herein can include administration of an effective concentration of GM-CSF or a pharmaceutical composition to a subject in need thereof resulting in an about 1% to about 100% (e.g., about 1%, about 5%, about 10%, about 20%, about 30%, about 40%, about 50%, about 60%, about 70%, about 80%, about 90%, up to 100%) increase in life expectancy compared to a control or non-treated subject or the state of the subject prior to administering the compositions described herein.

In some embodiments, methods disclosed herein can include administration of an effective concentration of GM-CSF or a pharmaceutical composition thereof to a subject in need thereof resulting in an about 1% to about 100% (e.g., about 1%, about 5%, about 10%, about 20%, about 30%, about 40%, about 50%, about 60%, about 70%, about 80%, about 90%, up to 100%) decrease in pancreatic islet amyloid deposition compared to a control or untreated subject or the state of the subject prior to administering the compositions described herein. In some embodiments, methods disclosed herein can include administration of an effective concentration of GM-CSF or a pharmaceutical composition thereof to a subject in need thereof resulting in an about 1% to about 100% (e.g., about 1%, about 5%, about 10%, about 20%, about 30%, about 40%, about 50%, about 60%, about 70%, about 80%, about 90%, up to 100%) decrease in amylin compared to a control or non-treated subject or the state of the subject prior to administering the compositions described herein.

In some embodiments, an effective concentration of GM-CSF or a pharmaceutical composition disclosed herein for use in a treatment regimen can be about 1 ug/m²/day to about 1 mg/m²/day, about 10 ug/m²/day to about 750 ug/m²/day, or about 250 ug/m²/day to about 500 ug/m²/day or about 250 ug/m²/day, or equivalent amount thereof. In some embodiments, an effective concentration of GM-CSF or a pharmaceutical composition disclosed herein for use can be about 250 ug/m²/day in a single, or in multiple doses. In certain embodiments, an effective concentration of GM-CSF or a pharmaceutical composition disclosed herein for use in a subject can be about 500 μg/m²/day. In certain embodiments, recombinant GM-CSF can be sargramostim, molgramostim, regramostim or other recombinant GM-CSF or a viral or plasmid vector or mRNA construct designed to encode GM-CSF and an effective concentration can be about 250 μg/m²/day to about 500 μg/m²/day or effective units of plasmid to see an effect administered in a single, or in multiple doses to a subject such as one-half dose given twice daily. In some embodiments, an effective concentration of sargramostim or other recombinant GM-CSF for use in treating a subject can be about 500 μg/m²/day or about 250 μg/m²/day. It is understood by one of skill in the art and/or a health professional that these ranges can be converted by standard methods to reflect treatment of mgs/kg.

In some embodiments, a GM-CSF or the like dosing regimen can include GM-CSF or compositions containing GM-CSF treatments of at least five days per week for up to three weeks. In accordance with these embodiments, treatments of GM-CSF can include treating the subject one time daily or more with a total dose of about 50 μg/m²/day to about 500 μg/m²/day; or about 100 μg/m²/day to about 300 pg/m²/day or about 125 μg/m²/day to about 250 pg/m²/day. In accordance with these embodiments, treatments of GM-CSF can include treating the subject with a daily dose of about 50 μg/m²/day to about 500 μg/m²/day intravenously over about 1-hour to about a 24-hour period: for example, by infusion. In accordance with these embodiments, treatments of GM-CSF can include treating the subject with a daily dose of about 50 μg/m²/day to about 500 μg/m²/day intravenously over about 1- hour, about a 2-hour, about a 4-hour, about a 6-hour, about a 12-hour, about an 18-hour, or about a 24-hour period. In accordance with these embodiments, treatments of GM-CSF can include treating the subject with a daily dose of about 50 μg/m²/day to about 500 μg/m²/day by at least one subcutaneous injection. In accordance with these embodiments, treatments of GM-CSF can include administering to the subject between at least one to at least three subcutaneous injections per day of about 50 μg/m²/day to about 500 μg/m²/day GM-CSF. In accordance with these embodiments, treatments of GM-CSF can include administering to the subject between about 50 μg/m²/day to about 500 μg/m²/day GM-CSF by continuous subcutaneous infusion using a portable pump. In other embodiments, treatments of GM-CSF can include administering to the subject continuously between about 50 μg/m²/day to about 500 μg/m²/day GM-CSF using a dissolvable GM-CSF-incorporated microneedle patch for predetermined delivery to the subject.

in certain embodiments, methods of the present disclosure can be used to personalize a GM-CSF dosing regimen recommendation for a subject having or suspected of developing at least one pathologic entity associated with Type 2 diabetes also referred to as T2DM (e.g., pancreatic islet amyloid deposition). In other embodiments, methods disclosed herein can ilicitide an initial GM-CSF dosing regimen or a GM-CSF-containing pharmaceutical composition disclosed herein given to a subject for a first course of treatment of 5 days per week for a pre-determined period (e.g., about a week to about a month), which can be followed by one or more GM-CSF dosing regimen.

In some embodiments, a subject to be treated by the method described herein can be a human subject who has undergone or is undergoing at least one other therapy for T2DM. In certain embodiments, the prior therapy for T2DM can be complete. In other embodiments, the other therapy for T2DM can be ongoing. In some embodiments, the subject can include a subject undergoing a combined therapy including a GM-CSF or recombinant GM-C SF (e.g., sargramostim, molgramostim, regramostim) disclosed herein and another therapy. In other embodiments, the subject can include a subject undergoing a combined therapy including exogenous administration of a viral or plasmid vector or mRNA construct encoding GM-C SF, or an analog, or biologically active fragment or mimetic thereof within the body of the subject being treated can be administered for short term or prolonged treatment and further include a second therapy for T2DM. Other therapies for Type 2 diabetes can include, but are not limited to, diet, exercise, one or more medications, dialysis, insulin therapy, or any combination thereof. Examples of agents of use to treat T2DM can include, but are not limited to, metformin, sulfonylureas, glitazones (e.g., pioglitazone), glinides (also referred to as “meglitinides”) (e.g., nateglinide, repaglinide), gliptins (also referred to as “dipeptidyl peptidase-4 inhibitors”) (e.g., linagliptin, saxagliptin, vildagliptin, sitagliptin), gliflozins (also referred to as “sodium—glucose cotransporter Type 2 inhibitors” or “SGLT2 inhibitors”) (e.g., dapagliflozin, empagliflozin, canagliflozin), alpha-glucosidase inhibitors (e.g., carbose), incretin mimetics (e.g., liraglutide, dulaglutide, lixisenatide, semaglutide, exenatide, albiglutide, tirzepatide), dipeptidyl peptidase-4 (DPP-4) inhibitors (e.g., sitagliptin, saxagiiptin, linagliptin, alog(ipiin), and the like. It is contemplated that these agents could be combined with GM-CSF therapies disclosed herein to treat a subject in a single composition, alternating dosing or other personalized treatment of the subject.

In certain embodiments, the present disclosure provide kits for use herein. A kit for therapeutic use as described herein can include one or more containers including GM-CSF (e.g., sargramostim, molgramostim, regramostim or vector for expressing GM-CSF or analog or mimetic thereof). In some embodiments, the kit can additionally have instructions for use of GM-CSF (e.g., sargramostim, molgramostim, regramostim) in any of the methods described herein. The instructions can include a description of storage, transport and/or administration of GM-CSF (e.g., sargramostim, molgramostim, regramostim) or a pharmaceutical composition to a subject to achieve the intended activity in a subject. The kit can further include a description of selecting a subject suitable for treatment based on identifying whether the subject is in need of the treatment. In some embodiments, the instructions can include a description of administering GM-CSF (e.g., sargramostim, molgramostim, regramostim) or the pharmaceutical composition to a subject who has or is suspected of developing at least one pathologic entity of Type 2 diabetes (e.g., pancreatic islet amyloid deposition).

In some embodiments, kits disclosed herein can include instructions relating to the use of GM-CSF (e.g., sargramostim, molgramostim, regramostim) or the pharmaceutical composition according to the methods disclosed herein. In some embodiments, instructions within the kit can generally include information as to dosage, dosing schedule, and route of administration for the intended treatment. In some embodiments, instructions of kits can include a description of optimizing the dose of GM-CSF (e.g., sargramostim, molgramostim, regramostim) in a subject having at least one pathologic entity of Type 2 diabetes (e.g., pancreatic islet amyloid deposition).

In some embodiments, the kit can have one or more containers. In some embodiments, containers can be unit doses, bulk packages (e.g., multi-dose packages) or sub- unit doses. In some embodiments, instructions supplied in kits of the disclosure can be written instructions on a label or package insert. In some embodiments, the label or package insert can indicate that the pharmaceutical compositions herein are used for treating, delaying the onset, and/or alleviating a disease or disorder in a subject.

In certain embodiments, kits provided herein are in suitable packaging. Suitable packaging includes, but is not limited to, vials, bottles, jars, flexible packaging, and the like. In some embodiments, kits can have packages for use in combination with a specific device, such as an inhaler, nasal administration device, or an infusion device. In some embodiments, a kit can have a sterile access port (for example, the container may be an intravenous solution bag or a vial having a stopper pierceable by a hypodermic injection needle). In some embodiments, a container can also have a sterile access port.

In certain embodiments, kits herein can provide additional components such as buffers and interpretive information. In some embodiments, a kit herein can have a container and a label or package insert(s) on or associated with the container. In some embodiments, the disclosure provides articles of manufacture comprising contents of the kits described above. EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of the invention. Example 1

In one exemplary method, a study was performed in a transgenic rodent model of Type 2 diabetes. Transgenic murine models required the expression of human genes. The transgenic hIAPP mouse model (also called RIPHAT) was used to determine whether GM-CSF could prevent and/or decrease pancreatic amyloid in RIPHAT mice, as compared to placebo (sterile saline)-treated control RIPHAT mice of the same age. Hemizygous RIPHAT mice, which demonstrated that pancreatic islets from 18-week-old male hemizygous RIPHAT mice do develop amylin fibrils, and which amylin fibrils were able to be reduced by Epigallocatechin Gallate (EGCG) treatment, were selected, whereas homozygous RIPHAT mice had noticeably smaller sized and lesser number of islets, which contained much less staining for amylin fibrils and which was not significantly reduced by EGCG treatment. Interestingly, in the hemizygous mice, islet numbers and insulin staining were not reduced, or blood glucose increased compared to wild-type mice and opposite from homozygous mice. However, the greater presence of pancreatic amylin fibrils within the hemizygous RIPHAT mice and the ability for a therapeutic to modify these amyloid levels make the hemizygous RIPHAT mice more appropriate than homozygous mice for testing GM-CSF's effects on pancreatic amyloid. Furthermore, pancreatic amyloid deposition is known to occur before f3-cell death in Type 2 diabetic patients. Another reason for using hemizygous RIPHAT mice, as opposed to homozygous mice, is because the disease phenotype in homozygous mice is too aggressive and fast progressing, beginning at 4-8 weeks of age until death by 16 weeks of age, which would not provide adequate treatment window for studying GM-CSF as a potential therapeutic. In this study, the transgenic mouse strain FVB/N-Tg(Ins2-IAPP)RHFSoel/J (also known as RIPHAT mice) was used as indicated above. RIPHAT transgenic mice express human islet amyloid polypeptide (h-IAPP) under the regulatory control of the rat insulin II promoter and were useful in studying the beta-cell destruction and islet amyloid deposition associated with non-insulin-dependent diabetes mellitus (NIDDM) or Type 2 diabetes mellitus (T2DM).

Sixteen hemizygous male RIPHAT mice at 12 weeks of age were used where the mice were divided into two sets of experiments. In the first set, there were 6 mice (2 GM-CSF treated, 4 saline treated), and in the second set, there were 10 mice (7 GM-CSF treated, 3 saline treated). To determine whether GM-CSF could potentially remove pancreatic amyloid, recombinant mouse GM-CSF was administered to the RIPHAT mice at a dose of 5 μg/day for each animal (average 30 grams each), which calculates to about twice the FDA-approved dosing of human GM-CSF/sargramostim (e.g. 250 μg/m 2 /day). The animals were treated with either a daily subcutaneous injection of GM-CSF (5 days/week) or saline for 23 injection days total, after which the animals were euthanized, perfused, and pancreas tissues obtained and processed for histochemical analyses.

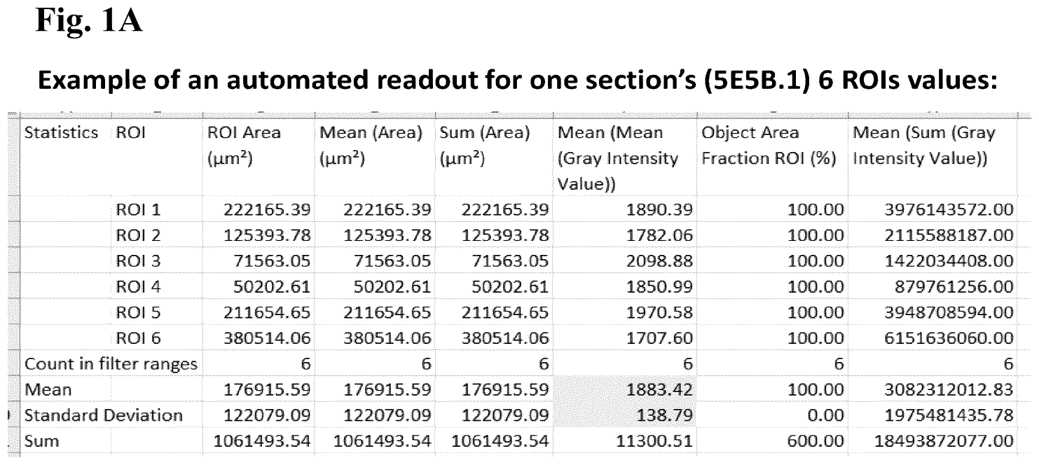

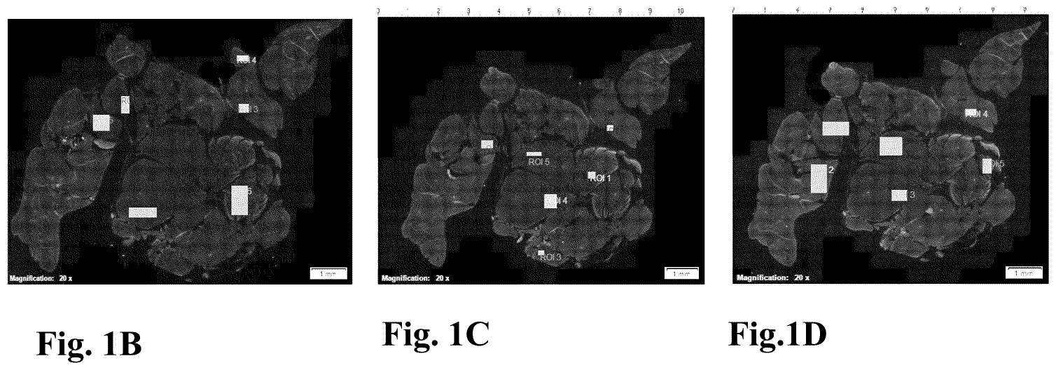

Paraffin-embedded tissues were sectioned and mounted on microscope slides with 3 tissue sections per slide. Using standard protocols, Thioflavin-S staining was performed to detect the presence of amyloid, with whole tissue images taken at 20X on an Olympus IX83 microscope with an automated motorized stage. To quantify Thioflavin-S staining, 3 tissue sections per mice were stained with Thioflavin-S and imaged. As illustrated in FIGS. 1B-1D, six independent regions of interest (ROIs) were selected on each section, with hyper-intense regions (e.g., edges, folds, debris artifacts) not being included in the ROIs. A staining threshold was set on a first representative section, and this threshold was applied and used to analyze intensity values for all 6 ROIs within each mouse's 3 sections (FIG. 1A) and across all mice. The mean and the standard deviations of each section's 6 ROI mean intensity values were compiled for overall analyses.

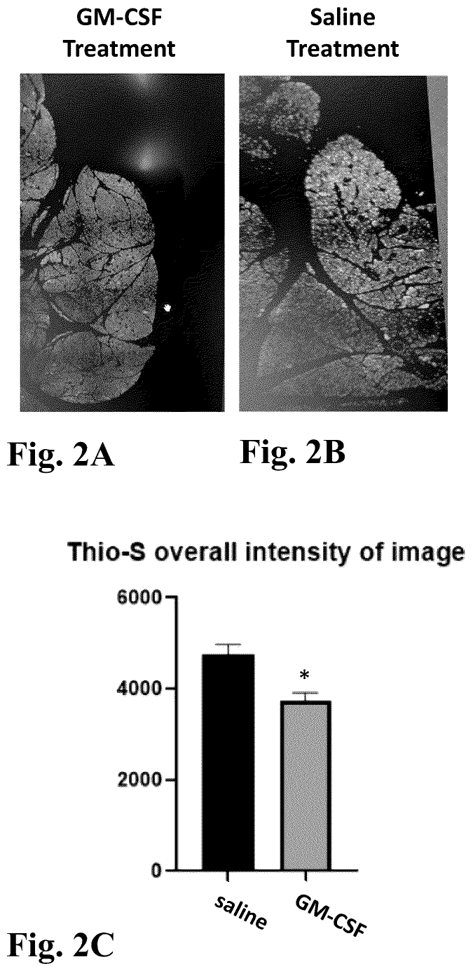

In the first set of animals (2 GM-CSF treated, 4 saline treated), there was a significantly lesser amount of pancreatic amyloid in the GM-CSF group compared to the saline group (p=0.0140) (FIG. 2C). Visual observation on this effect was seen with more Thioflavin-S staining in the saline-treated picture (FIG. 2B) compared to the GM-CSF-treated picture (FIG. 2A). In the second set of animals (7 GM-CSF treated, 3 saline treated), it was also found that there was a significantly less amount of pancreatic amyloid in the GM-CSF group as compared to the saline group (p=0.00114) by standard Excel TTest analysis (2 sided, unequal variance).

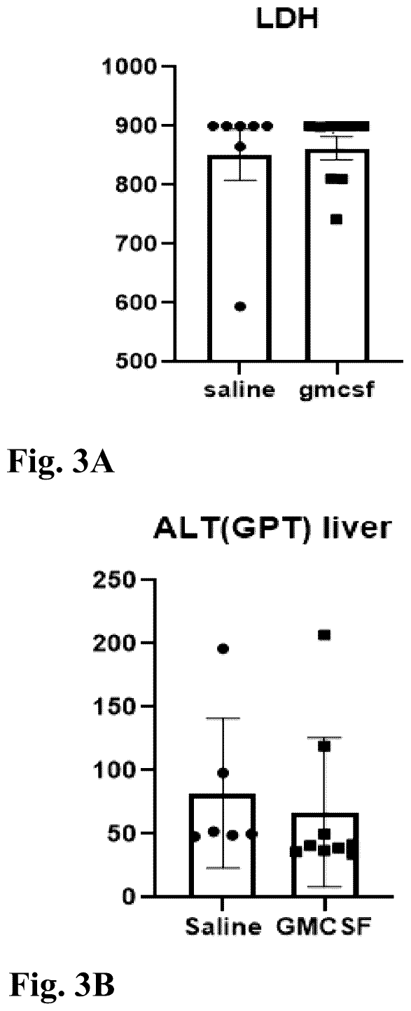

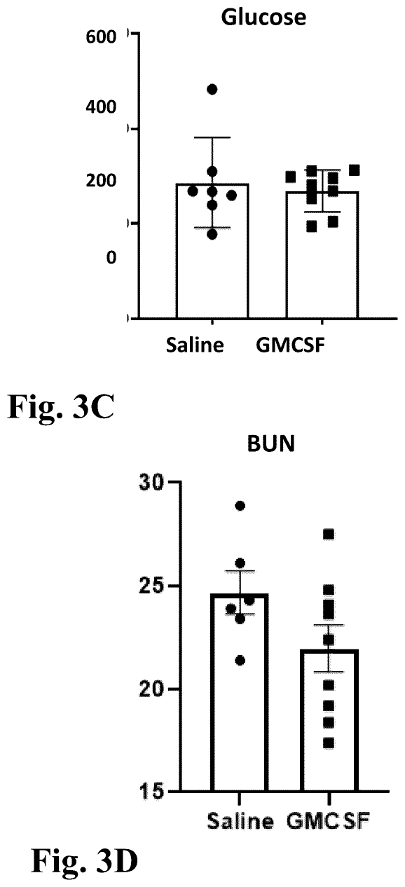

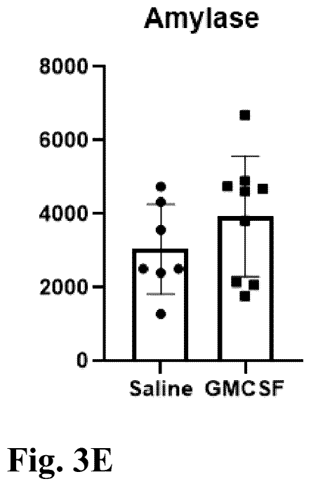

Whole blood samples were also obtained from each animal before perfusion, and blood chemistry analyses for selected T2DM-related biomarkers [lactate dehydrogenase (LDH) (FIG. 3A), alanine transaminase (ALT) (FIG. 3B), glucose (FIG. 3C), BUN (FIG. 3D), and amylase (FIG. 3E)]. Blood chemistry results did not demonstrate statistically significant differences between groups. It was known that hemizygous RIPHAT mice do not demonstrate changes in blood glucose increased compared to wild-type mice. Although significance was not reached, some noticeable trends in the collected blood chemistries were observed. Specifically, BUN levels, which are typically increased in T2DM, were decreased in GM-CSF-treated hemizygous RIPHAT mice compared to saline-treated hemizygous RIPHAT mice (FIG. 3D). Also, amylase levels, which are typically decreased in T2DM, were increased in GM-CSF-treated hemizygous RIPHAT mice compared to saline-treated hemizygous RIPHAT mice (FIG. 3E). Example 2

### Caspase Study

In another exemplary method, a total of 16 hemizygous male RIPHAT mice at 12 weeks of age were used, which were divided into two sets of experiments. In the first set, there were 6 mice (2 GM-CSF treated, 4 saline treated), and in the second set, there were 10 mice (7 GM-CSF treated, 3 saline treated). In designing this study to determine whether GM-CSF could potentially be a therapeutic against Type 2 Diabetes mellitus by removing pancreatic amylin amyloid, preventing pancreatic cell death, and preserving pancreatic function, it was decided to use recombinant mouse GM-CSF at a dose of 5μg/day (approximately: 167 μg/kg as is readily convertible by one of skill in the relevant art) for each animal (average 30 g each), which is the same dosage used in mouse models of other diseases, such as pre-clinical Alzheimer's disease study (previously reported), and which calculates to about twice the FDA-approved human equivalent dosing of recombinant human GM-CSF/sargramostim (e.g., 250μ/m 2 /day). The animals were treated with either a daily subcutaneous injection of GM-CSF (5 days/week) or saline for 23 injection days total, after the animals were euthanized and perfused according to regulations, and pancreas tissues obtained and processed for histochemical analyses.

| Unpaired t test | |
|---|---|
| P value | 0.0057 |
| P value summary | ** |
| Significantly different (P < 0.05)? | Yes |
| One- or two-tailed P value? | Two-tailed |
| t, df | t = 2.903, df = 46 |

Immunohistochemistry (IHC): Paraffin-embedded tissues were sectioned and mounted on microscope slides. Using standard protocols, we performed Caspase-3 staining for evidence of cells undergoing apoptosis. Specifically, three tissue sections per animal were stained with a primary monoclonal Caspase-3 antibody (Thermo Fisher: CPP32 4-1-18; dilution 1:100), followed by secondary antibody staining using Alexa Fluor® 594 (Invitrogen: A-11032; dilution 1:2000) and lastly using ProLongTM Diamond Antifade Mountant with DAPI (ThermoFisher: P36971) mounting media according to manufacturer instructions. Each tissue section was then imaged as whole tissue images taken at 20X on an Olympus IX83 microscope with an automated motorized stage and followed by image analyses as follows.

Imaging analyses: (the same techniques were used as illustrated for Thio-S staining analyses as illustrated in FIGS. 1B-1D). Specifically: 6 independent Regions of Interest (ROIs) were selected on each imaged section, with hyper-intense regions (eg., edges, folds, debris artifacts) not being included within the ROIs. A staining threshold was set on a first representative section, and then this threshold was applied and used to analyze intensity values for all 6 ROIs within each animal's 3 sections and across all animals. The Mean and standard deviation of each section's 6 ROI mean intensity values were compiled for overall analyses.

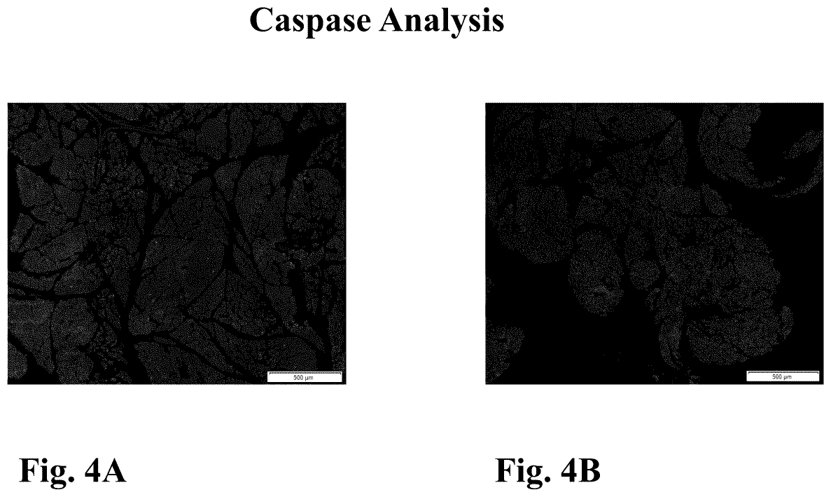

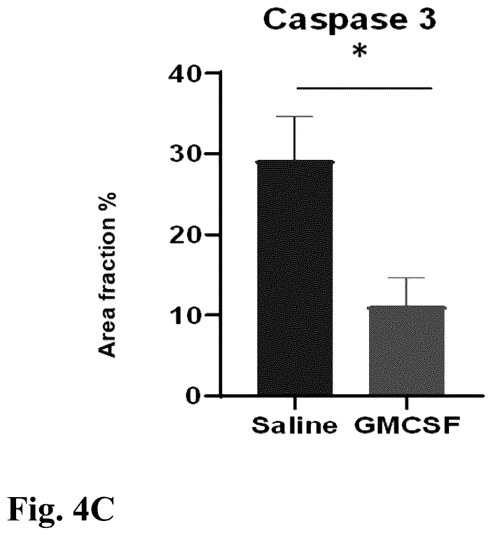

Using PRISM statistical analyses of these studies, significantly less Caspase-3 staining was observed in the GM-CSF group compared to the saline control group (p=0.0057), indicating that the saline-treated group had more pancreatic cells undergoing apoptosis and that GM-CSF treatment reduced or prevented programmed cell death in the pancreas. Visual observation on this effect can be seen in the images above with more Caspase-3 staining (red color, FIGS. 4A and 4B) in the saline-treated picture, as compared to the GM-C SF-treated picture. These data demonstrate that GM-C SF is preventing pancreatic cell death in treated mice, when compared to saline-treated mice not receiving GM-CSF (FIG. 4C) indicating that these GM-C SF treatments can preserve pancreatic islet cells and reduce apoptosis in this acceptable model.

All of the COMPOSITIONS and METHODS disclosed and claimed herein can be made and executed without undue experimentation in light of the present disclosure. While the COMPOSITIONS and METHODS have been described in terms of preferred embodiments, it will be apparent to those of skill in the art that variation can be applied to the COMPOSITIONS and METHODS and in the steps or in the sequence of steps of the METHODS described herein without departing from the concept, spirit and scope of the invention. More specifically, it will be apparent that certain agents which are both chemically and physiologically related can be substituted for the agents described herein while the same or similar results would be achieved. All such similar substitutes and modifications apparent to those skilled in the art are deemed to be within the spirit, scope and concept of the invention as defined by the appended claims. WHAT IS CLAIMED IS:

1. A method of reducing the risk of, preventing, or treating a pathologic entity of Type 2 diabetes in a subject, the method comprising administering to the subject a composition comprising granulocyte macrophage colony stimulating factor (GM-CSF) or an mRNA construct encoding GM-CSF, recombinantly produced molecule thereof, or biologically active fragment thereof, or an analog thereof wherein the subject has or is suspected of developing a pathologic entity of Type 2 diabetes.

2. The method according to claim 1, wherein the pathologic entity of Type 2 diabetes in the subject is pancreatic islet amyloid depositions.

3. The method according to claim 1, wherein the GM-CSF comprises synthetic GM-CSF, recombinantly produced GM-CSF, naturally-occurring GM-CSF or a vector expressing GM-CSF, or an mRNA construct encoding GM-CSF.

4. The method according to claim 1, wherein the GM-CSF comprises sargramostim, molgramostim, or regramostim.

5. The method according to claim 1, wherein administering the composition comprises administering the composition comprising about 10 μg/m²/day to about 1,000 μg/m²/day of GM-CSF, recombinantly produced molecule thereof, or biologically active fragment thereof, or an analog thereof to the subject.

6. (canceled)

7. The method according to claim 1, wherein the composition reduces pancreatic islet amyloid deposition in the subject.

8. The method according to claim 1, wherein administering the composition reduces blood urea nitrogen (BUN) levels in the subject relative to the level of BUN of the subject prior to administration of the composition.

9. The method according to claim 1, wherein administering the composition increases serum amylase levels in the subject.

10. The method according to claim 1, wherein the composition comprises a pharmaceutical composition and further comprises a pharmaceutically acceptable carrier or excipient.

11. The method according to claim 1, wherein administering the composition comprises administering the composition intravenously, subcutaneously, intradermally, by inhalation, or orally.

12. The method according to claim 1, wherein the subject is a human subject.

13. A method of preventing, reducing, or treating amyloid depositions in a subject having a condition, the method comprising administering to the subject a composition comprising granulocyte macrophage colony stimulating factor (GM-CSF), recombinantly produced molecule thereof, or biologically active fragment thereof, or an analog thereof, wherein the subject has amyloid depositions or is suspected of developing amyloid depositions.

14. The method according to claim 13, wherein the GM-CSF comprises synthetic GM-CSF, recombinantly produced GM-CSF, naturally-occurring GM-CSF or a vector expressing GM-CSF.

15. (canceled)

16. The method according to claim 13, wherein the subject further comprises a subject having at least one metabolic risk factor.

17. The method according to claim 16, wherein the at least one metabolic risk factor comprises abdominal obesity, hypertension, dyslipidemia, hyperinsulinemia, or any combination thereof.

18. The method according to any one of claims 13-17 claim 13, wherein the subject comprises a pre-Type 2 diabetic subject or a Type 2 diabetic subject.

19-20 (canceled)

21. The method according to claim 13, wherein the amyloid deposits comprise one or more of pancreatic islet amyloid deposits, heart amyloid deposits, kidney amyloid deposits, antibody deposits in any organ, transthyretin, apoprotein Al, and gelsolin deposits, SAA (senile systemic amyloid) deposits, β2-microglobulin accumulation, and cutaneous amyloid deposits or other amyloid deposits.

22. The method according to claim 13, wherein the subject is undergoing at least one other standard treatment for the condition.

23. A kit for treating a subject having or suspected of developing Type 2 diabetes comprising a composition comprising granulocyte macrophage colony stimulating factor (GM-CSF), recombinantly produced molecule thereof, or biologically active fragment thereof, or an analog thereof; and at least one container.

24. The kit according to claim 23, further comprising a device or composition for measuring blood glucose levels.
